(19) United States
(12) Patent Application Publication
(10) Pub. No.: US 2004/0266849 A1
(43) Pub. Date: Dec. 30, 2004

(54) Therapeutic compounds for treating dyslipidemic conditions

(76) Inventors: Alan D Adams, Cranford, NJ (US); Shaei Y Huang, Jeffersonville, PA (US); Bruno Tse, San Diego, CA (US); Ahren I Green, Riegelsville, PA (US); A Brian Jones, Arkesden (GB)

Correspondence Address: MERCK AND CO INC, P O BOX 2000, RAHWAY, NJ, 070650907

(21) Appl. No.: 10/498,771

(22) PCT Filed: Dec. 16, 2002
(86) PCT No.: PCT/US02/40236

(51) Int. Cl. C07D 261/20; A61K 31/42

(52) U.S. Cl. 514/379; 548/241

(57) ABSTRACT

The present invention relates to novel LXR ligands of Formula (I) and the pharmaceutically acceptable salts and esters thereof, which are useful in the treatment of dyslipidemic conditions, particularly depressed levels of HDL cholesterol.

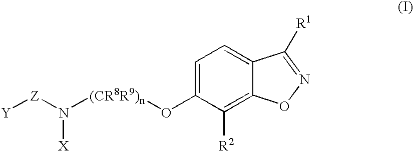

### BACKGROUND OF THE INVENTION

Recent publications in Nature Genetics, August, 1999 (Young et al, page 316; Bodzioch et al, page 347; Brooks-Wilson et al, page 335, and Rust et al, page 352 ) showed that humans with mutations in the gene ABCAI (also previously known in the art as ABC1) have low levels of high density lipoprotein (HDL). Low HDL levels are a risk factor for atherosclerosis, myocardial infarction and related conditions such as ischemic stroke. Therefore, increasing the expression of the ABCA1 gene would be expected to increase HDL levels and decrease the occurrence of atherosclerosis, myocardial infarction and related conditions such as ischemic stroke. It has been reported that expression of the ABCA1 gene is increased by cholesterol loading of cells (Langmann et al, Biochem. Biophys. Res. Comm., 257, 29-33 (1999)). LXRα is a nuclear receptor that is required for the induction of cholesterol 7α-hydroxylase in mouse liver following cholesterol feeding (Peet et al, Cell, 93, 693-704 (1998)). LXRα and LXRβ are activated by 22-(R)-hydroxycholesterol and other oxysterols (Janowski et al. Proc. Natl. Acad. Sci USA, 96, 266-271 (1999), Thomas A. Spencer et al. J. Med. Chem., 44, 886-897, (2001)). Some non-steroidal small molecule agonists of LXRα and LXRβ have been reported to affect circulating HDL levels, cholesterol absorption, reverse cholesterol transport and ABCA1 expression in vivo (J. R. Schultz, et al. Genes & Devel. 14, 2831-2838, (2000), J. J. Repa et al. Science, 289, 1524-1529, (2000)) It has been found that LXRα and/or LXRβ cause the induction or regulation of ABCA1 expression, and that small molecule ligands of LXR are useful as drugs to increase the expression of ABCA1, increase levels of HDL and thereby decrease the risk of atherosclerosis, myocardial infarction and related conditions such as peripheral vascular disease and ischemic stroke.

The various dyslipidemic conditions, which are risk factors for atherosclerosis, are currently treated with several different classes of drugs, such as statins which are HMG-CoA reductase inhibitors, bile acid sequestrants (e.g., cholestyramine and colestipol), nicotinic acid (niacin), and fibrates. However, except for niacin, most of these treatments do not raise HDL as their primary effect. With favorable outcomes in many human studies, the statin class of drugs is used to modulate LDL and, to a lesser extent, HDL and triglycerides. Conditions principally characterized by elevated plasma triglycerides and low HDL are frequently treated with drugs belonging to the fibrate class. The fibrates are PPAR alpha agonists that lower triglycerides and raise HDL in many instances. There are no currently marketed drugs whose principal actions are mediated by LXR.

We have now discovered a new class of small molecules which are LXR ligands, i.e., LXRα and/or LXRβ ligands, and are therefore expected to be useful for modulation of HDL levels, ABCA1 gene expression and reverse cholesterol transport. The instant compounds have been shown to raise plasma levels of HDL in animal models and to increase cholesterol efflux from cells in vitro. These biological activities are critical for reverse cholesterol transport.

The novel compounds of this invention are intended as a treatment for dyslipidemias, especially low plasma HDL cholesterol levels, as well as for treatment and/or prevention of lipid accumulation in atherosclerotic plaques, which is an underlying cause or aggravating factor in atherosclerosis.

### SUMMARY OF THE INVENTION

Compounds of Formula I are novel LXR ligands and are useful in the treatment of dyslipidemic conditions including below-desirable levels of HDL cholesterol.

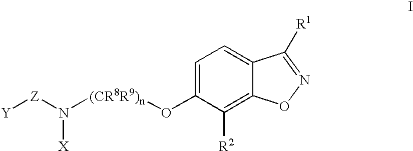

One object of the instant invention is to provide a method for treating depressed plasma HDL cholesterol levels comprising administering a therapeutically effective amount of a compound of Formula I to a patient in need of such treatment.

Another object is to provide a method for preventing or treating dyslipidemic conditions comprising administering a prophylactically or therapeutically effective amount, as appropriate, of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. The method of this invention also serves to remove cholesterol from tissue deposits such as xanthomas and atherosclerotic lesions by hastening the efflux of cholesterol from cells in those lesions. Additional objects will be evident from the following detailed description.

Other objects of this invention are to provide processes for making the compounds of Formula I and to provide novel pharmaceutical compositions comprising these compounds. Additional objects will be evident from the following detailed description.

### DETAILED DESCRIPTION OF THE INVENTION

The novel LXR ligands of the instant invention are compounds of Formula I

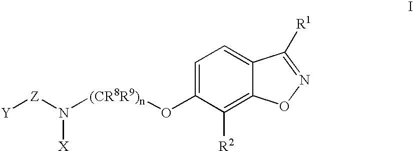

and the pharmaceutically acceptable salts and esters thereof, wherein

R¹ is selected from the group consisting of:

(a) —CF₃,

(b) —CH₂C(CH)₃,

(c) phenyl,

(d) -C₁₋₆ alkyl, and

(e) -C₁₋₂ alkyl-phenyl;

R² is selected from the group consisting of:

(a) -C₁₋₆ alkyl,

(b) —COOR³,

(c) —CR³R⁴—O—R⁵,

(d) —CR³R⁴—S—R⁵, and

(e) —COR³;

R³, R⁴ and R⁵ are independently selected at each occurrence from the group consisting of —H, phenyl, and C₁₋₆ alkyl;

n is an integer selected from 2, 3, 4, 5 and 6;

Z is selected from

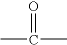

and —S(O)ₘ—, wherein m is an integer selected from zero, 1 and 2;

X is selected from the group consisting of:

(a) —H,

(b) —C(O)CH₃,

(c) -C₁₋₆alkyl, and

(d) —CH₂CF₃;

Y is selected from the group consisting of:

(a) -C₁₋₈ straight chain alkyl, unsubstituted, mono- or poly-substituted with a substituent independently selected at each occurrence from the group consisting of:

(i) —COOR³,

(ii) —CONH₂,

(iii) —CN,

(iv) halo,

(v) thienyl,

(vi) —S-phenyl,

(vii) tetrazolyl,

(viii) NR⁶R⁷,

(ix) phenyl, unsubstituted, mono- or poly-substituted with a substituent independently selected at each occurrence from the group consisting of: —COOR³, halogen and C₁₋₆alkyl,

(x) -C₁₋₃alkyl, unsubstituted, mono- or poly-substituted with a substituent independently selected at each occurrence from the group consisting of: —COOR³, halogen, and -C₁₋₃alkyl,

(b) phenyl, unsubstituted, mono- or poly-substituted with a substituent independently selected at each occurrence from the group consisting of: —COOR³, halogen and -C₁₋₆alkyl,

(c) furanyl, unsubstituted, mono- or poly-substituted with a substituent independently selected at each occurrence from the group consisting of: —COOR₃, halogen and -C₁₋₆alkyl,

(d) pyridyl, unsubstituted, mono- or poly-substituted with a substituent independently selected at each occurrence from the group consisting of: —COOR³, halogen and -C₁₋₆alkyl,

(e) thienyl, unsubstituted, mono- or poly-substituted with a substituent independently selected at each occurrence from the group consisting of: —COOR³ halogen, and -C₁₋₆alkyl,

(f) -C₃₋₆cycloalkyl, unsubstituted, mono- or poly-substituted with a substituent independently selected at each occurrence from the group consisting of: —COOR³, halogen and -C₁₋₆alkyl, and

(g) -C₁₋₄alkyl-O-C₁₋₄alkyl, provided that the total number of carbons is from 3 to 5, unsubstituted, mono- or poly-substituted with a substituent independently selected at each occurrence from the group consisting of: —COOR³ and -C₁₋₆alkyl;

R⁶ is selected from the group consisting of —H and -C₁₋₆alkyl;

R⁷ is selected from the group consisting of:

(a) —COC₁₋₃ alkyl,

(a) —COCF₃, and

(b) —COOR³;

R⁸ and R⁹ are independently selected at each occurrence from the group consisting of —H, C₁₋₄ alkyl, -C₁₋₄ alkenyl, —O-C₁₋₄ alkyl and —O-C₁₋₄ alkenyl;

provided that when Z is —S(O)ₘ—, then Y is -C₁₋₈ straight chain alkyl; and

provided that when Z is —C(O)—, X is H and Y is phenyl substituted with -C₁₋₂ alkyl, then the C₁₋₂ alkyl is not substituted with —COOR³ on the terminal carbon.

In one embodiment of this invention are compounds of Formula I wherein Z is —C(O)—.

In one class of the first embodiment are those compounds of Formula I wherein R¹ is selected from —CF₃ and —CH₂C(CH)₃ and R² is n-propyl.

In a second class of the first embodiment are compounds of Formula I wherein X is selected from —H and -C₁₋₆alkyl, and n is selected from 3, 4 and 5. In a sub-class of the second class are compounds wherein R¹ is selected from —CF₃ and —CH₂C(CH)₃ and R² is n-propyl.

In a third class of the first embodiment are compounds of Formula I wherein Y is selected from

(a) -C₁₋₈ straight chain alkyl, unsubstituted, mono- or poly-substituted with a substituent independently selected at each occurrence from the group consisting of:

(i) —COOR³,

(ii) —CONH₂,

(iii) —CN,

(iv) -halo,

(v) —S-phenyl,

(vi) tetrazolyl, and

(vii) C₁₋₃alkyl, unsubstituted, mono- or poly-substituted with a substituent independently selected at each occurrence from the group consisting of: —COOR³, halogen, and -C₁₋₃alkyl,

(b) phenyl, unsubstituted, mono- or poly-substituted with a substituent independently selected at each occurrence from the group consisting of: —COOR³, halogen and -C₁₋₆alkyl,

(c) furanyl, unsubstituted, mono- or poly-substituted with a substituent independently selected at each occurrence from the group consisting of: —COOR³, halogen and -C₁₋₆alkyl,

(d) pyridyl, unsubstituted, mono- or poly-substituted with a substituent independently selected at each occurrence from the group consisting of: —COOR³, halogen and -C₁₋₆alkyl,

(e) thienyl, unsubstituted, mono- or poly-substituted with a substituent independently selected at each occurrence from the group consisting of: —COOR³, halogen and -C₁₋₆alkyl, and

(f) -C₃₋₆cycloalkyl, unsubstituted, mono- or poly-substituted with a substituent independently selected at each occurrence from the group consisting of: —COOR³, halogen and -C₁₋₆alkyl.

In sub-class (i) of the third class are compounds wherein R¹ is selected from —CF₃ and —CH₂C(CH)₃ and R² is n-propyl.

In sub-class (ii) of the third class are compounds wherein R¹ is selected from —CF₃ and —CH₂C(CH)₃, R² is n-propyl, X is selected from —H, —C(O)CH₃, and -C₁₋₃alkyl, R⁸ and R⁹ are independently selected from H and —O-C₁₋₄ alkenyl, and n is selected from 3, 4 and 5. Specific examples within subclass (ii) of the third class are defined for Formula I in Table 1.

TABLE 1

| | |
|---|---|
| (1a) | 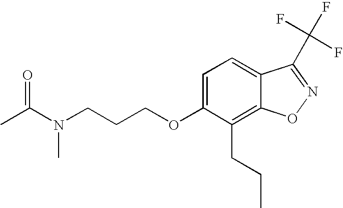 |
| (1b) | 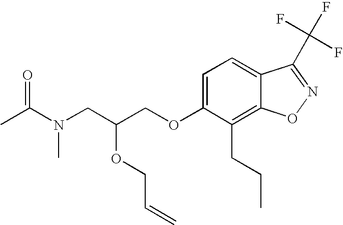 |
| (1c) | 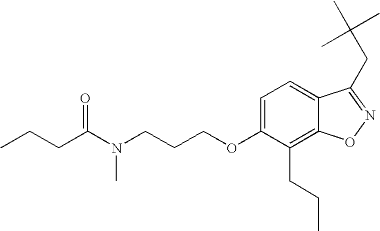 |
| (1d) | 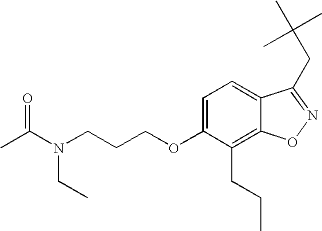 |
| (1e) | 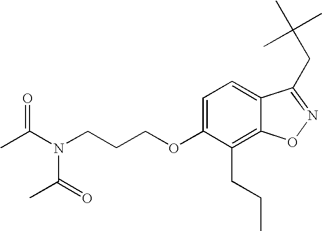 |
| (1f) | 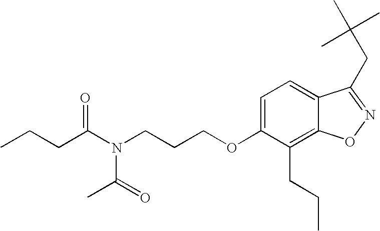 |
| (1g) | 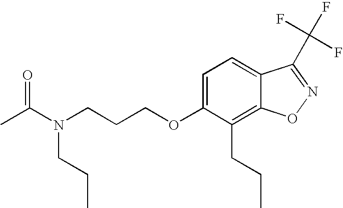 |
| (1h) | 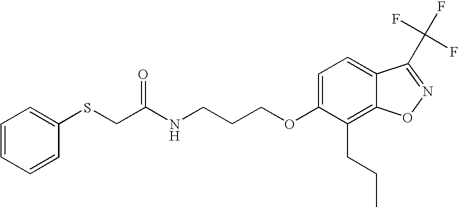 |
| (1i) | 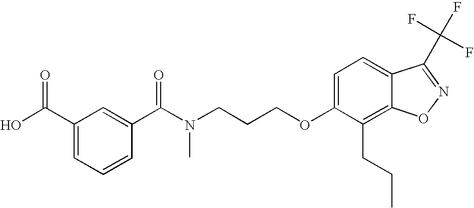 |
| (1j) | 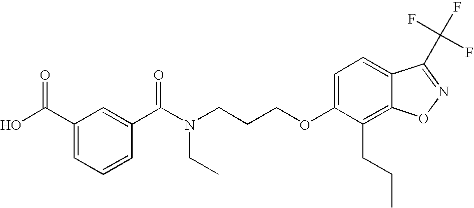 |
| (1k) | 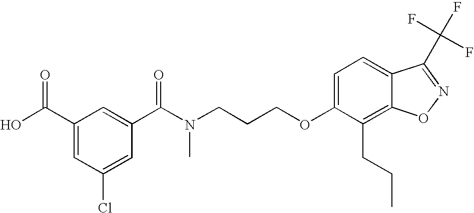 |
| (1l) | 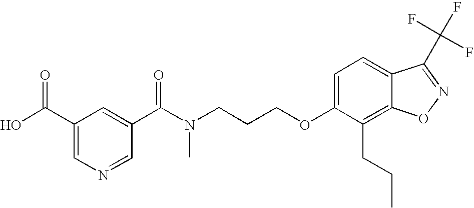 |
| (1m) | 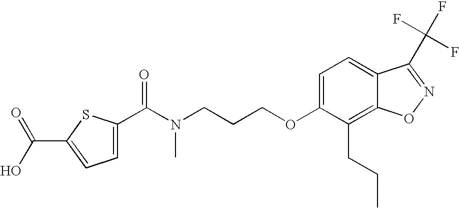 |
| (1n) | 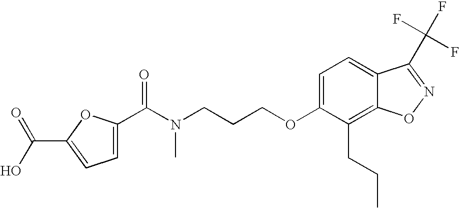 |
| (1o) | 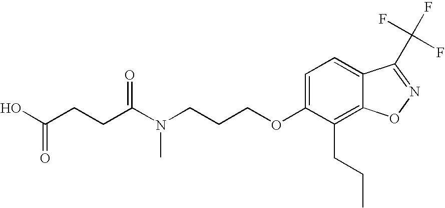 |
| (1p) | 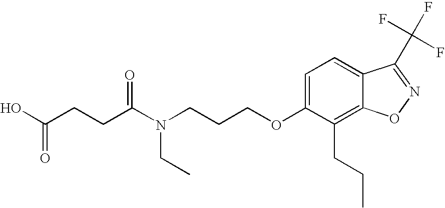 |
| (1q) | 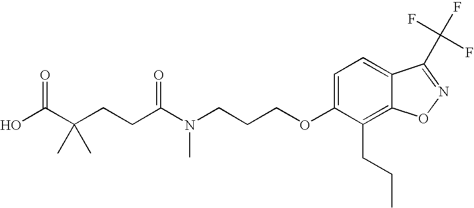 |
| (1r) | 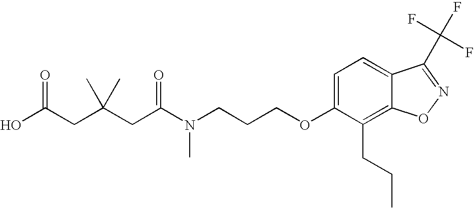 |
| (1s) | 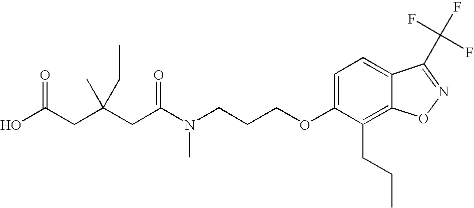 |
| (1t) | 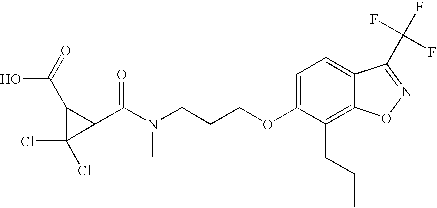 |
| (1u) | 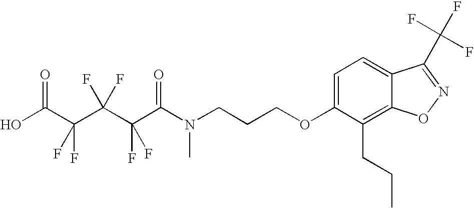 |
| (1v) | 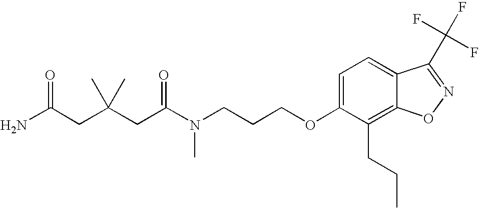 |
| (1w) | 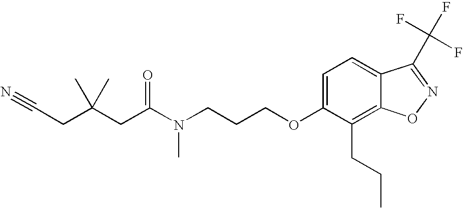 |
| (1x) | 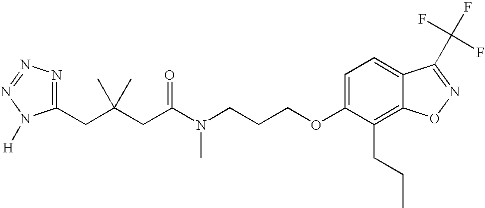 |

In sub-class (iii) of the third class are compounds of Formula I wherein R¹ is —CF₃; R² is n-propyl; X is selected from —H and -C₁₋₂alkyl; n is selected from 3 and 4; R⁸ and R⁹ are H; and Y is selected from

(a) -C₁₋₈ straight chain alkyl, unsubstituted, mono- or poly-substituted with a substituent independently selected at each occurrence from —COOH, —CN, tetrazolyl, and -C₁₋₃alkyl,

(b) phenyl, unsubstituted or mono- or poly-substituted with —COOH,

(c) pyridyl, unsubstituted or mono- or poly-substituted with —COOH,

(d) thienyl, unsubstituted or mono- or poly-substituted with —COOH, and

(e) -C₃₋₆cycloalkyl, unsubstituted, mono- or poly-substituted with a substituent independently selected at each occurrence from —COOH and halogen.

Specific examples within subclass (iii) of the third class are defined for Formula I in Table 2.

TABLE 2

| | |
|---|---|
| (2a) | 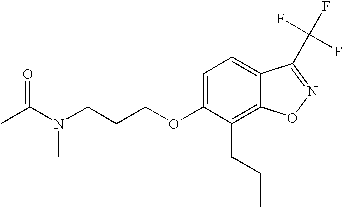 |
| (2b) | 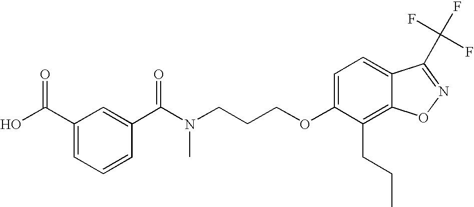 |
| (2c) | 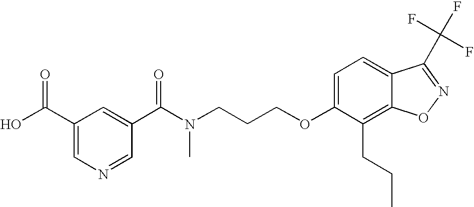 |
| (2d) | 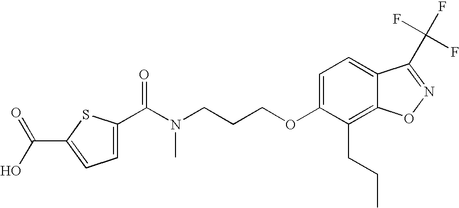 |
| (2e) | 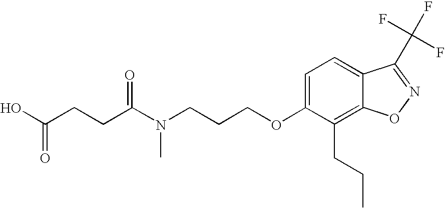 |
| (2f) | 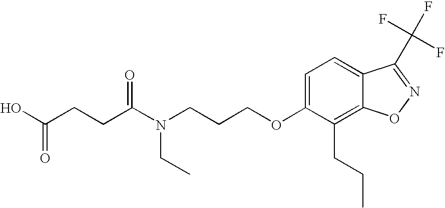 |
| (2g) | 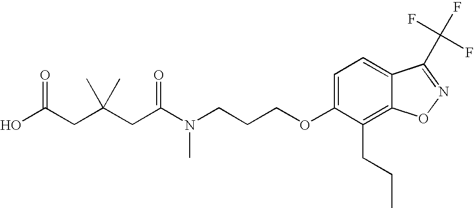 |
| (2h) | 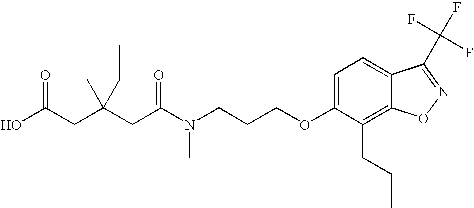 |
| (2i) | 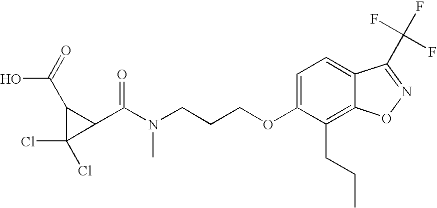 |
| (2j) | 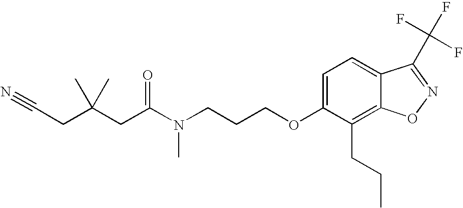 |
| (2k) | 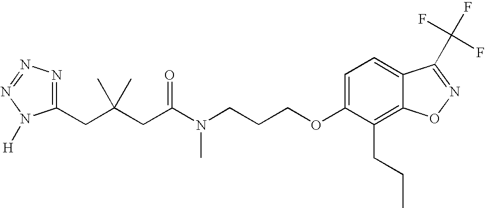 |

In sub-class (iv) of the third class are compounds of Formula I wherein R¹ is —CF₃; R² is n-propyl; n is 3; X is methyl; R⁸ and R⁹ are H; and Y is selected from

(a) -C₁₋₈ straight chain alkyl, unsubstituted, mono- or poly-substituted with a substituent independently selected at each occurrence from —COOH and -C₁₋₃alkyl,

(b) phenyl, unsubstituted or mono- or poly-substituted with —COOH, and

(c) -C₃₋₆cycloalkyl, unsubstituted, mono- or poly-substituted with a substituent independently selected at each occurrence from —COOH and halogen.

Specific examples within subclass (iv) of the third class are defined for Formula I in Table 3.

TABLE 3

| | |
|---|---|
| (3a) | 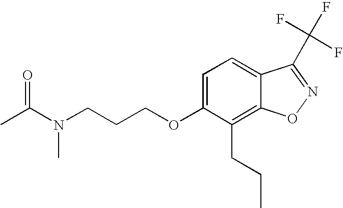 |
| (3b) | 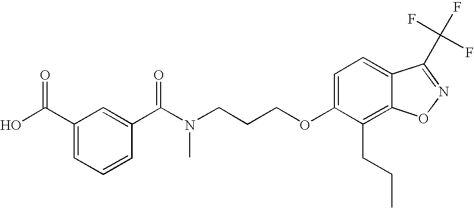 |
| (3c) | 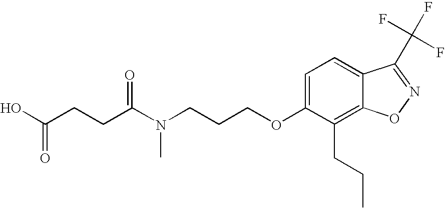 |
| (3d) | 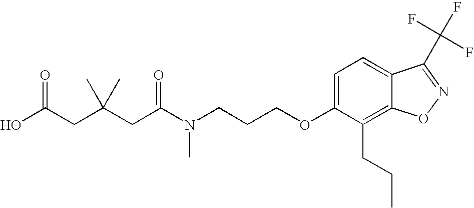 |
| (3e) | 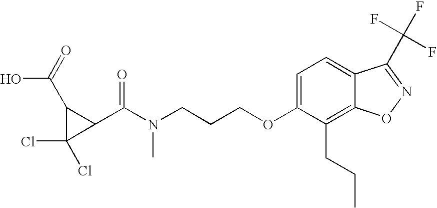 |

In a second embodiment of this invention are compounds of Formula I wherein Z is —S(O)ₘ—. In one class of this embodiment are those compounds of Formula I wherein m is 2. Specific examples of the second embodiment of this invention are defined for Formula I in Table 4.

TABLE 4

| | |
|---|---|
| (4a) | 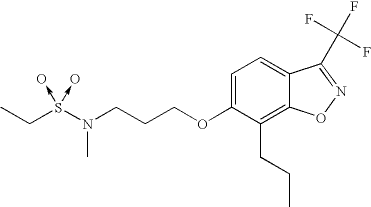 |
| (4b) | 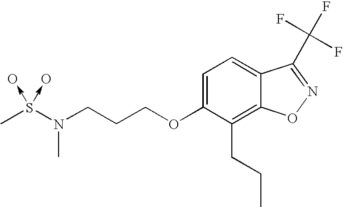 |

As used herein “alkyl”, except where specifically noted otherwise, is intended to include both branched- and straight-chain saturated aliphatic hydrocarbon groups having the specified number of carbon atoms, e.g., methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), isopentyl, isohexyl and the like. Alkyl groups are unsubstituted or optionally substituted where noted herein.

The term “C₁₋₄ alkenyl” as used herein, refers to a straight or branched 1-4 carbon chain with at least one carbon-carbon double bond. The double bond may be formed between the alkenyl group carbon and the carbon to which the alkenyl group is attached. For example, a double bond may be formed between the carbon in the C₁₋₈ straight chain alkyl group to which the alkenyl group is attached and the adjacent carbon of the alkenyl group. Suitable examples of alkenyl substituents include, but are not limited to, the following: ═CH₂, ═CH—CH₃, ═CH—CH₂—CH₃, —CH═CH₂, —CH═CH—CH₃, —CH₂—CH═CH₂, —CH═CH—CH₂—CH₃, —CH₂—CH═CH—CH₃, —CH₂—CH₂—CH═CH₂, ═CH₂, and ═CH—CH₃.

The term halo or halogen is meant to include fluoro, chloro, bromo and iodo, unless otherwise noted. Fluoro is preferred.

The term “thienyl,” as used herein, refers to the group

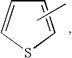

Herein, the term “pharmaceutically acceptable salts” shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, omithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)aminomethane. Pharmaceutically acceptable esters at the carboxylic acid group can be made by treating a dihydroxy open acid statin with an alcohol. Examples of pharmaceutically acceptable esters of dihydroxy open acid statins include, but are not limited to, -C₁₋₄ alkyl and - C₁₋₄ alkyl substituted with phenyl-, dimethylamino-, and acetylamino. “C₁₋₄alkyl” herein includes straight or branched aliphatic chains containing from 1 to 4 carbon atoms, for example methyl, ethyl, n-propyl, n-butyl, iso-propyl, sec-butyl and tert-butyl.

When referring to moieties which may optionally be substituted herein, e.g., alkyl groups, cyclopropyl groups, phenyl groups, heterocyclic groups and the like, the phrase used herein “independently unsubstituted, mono- or poly-substituted with a substituent independently selected at each occurrence” is intended to mean that the total number of substituents on the moiety overall may be zero, one or more than one, and that each carbon atom that is available for substitution in the given moiety may independently be unsubstituted, or mono- or poly-substituted, with one or more substituents that are the same or different at each occurrence and which result in the creation of a stable structure. The term “poly-substituted” is intended to.mean two or more substitutions, e.g. di-, tri-, tetra-, penta- substitution, and higher as appropriate.

In choosing compounds of the present invention, one of ordinary skill in the art will recognize that the various substituents, i.e. R¹, R², R³, etc., are to be chosen in conformity with well-known principles of chemical structure connectivity. When any variable (e.g., R³, R⁴, etc.) occurs more than one time in any constituent or in formula I, its definition on each occurrence is independent of its definition at every other occurrence. Also, combinations of substituents and/or variables are permissible only if such combinations result in stable compounds.

The compounds of the present invention may be chiral and the present compounds may occur as diasteriomeric mixtures, racemates (racemic mixtures) and as individual diasteriomers or enantiomers with all such isomeric forms being included within the scope of this invention, except where the stereoconfiguration of a specific chiral center is defined or depicted otherwise. Some of the compounds described herein contain olefinic double bonds, and unless specified otherwise, are meant to include both E and Z geometric isomers. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates are encompassed within the scope of this invention.

Some abbreviations used herein are as follows: Ac is acetyl [CH₃C(O)—]; PG is protecting group; Ph is phenyl; PhMe is toluene; Bn is benzyl; DMF is N,N-dimethylformamide; DMSO is di-methyl sulfoxide; THF is tetrahydrofuran; TMS is trimethylsilyl; HOBt is 1-hydroxybenzotriazole; EDAC (or EDC) is 1-ethyl-3-[3-(dimethylamino)propyl]carbodiimide HCl; HCl is hydrochloric acid; NaHMDS is sodium hexamethyldisiliazide, DIBAL is diisobutylaluminum hydride; TPAP is tetrapropylammonium perruthenate; NMO is N-methylmorpholine N oxide; HPLC is high performance liquid chromatography; TLC is thin layer chromatography; RT is ambient temperature; N is normal; mmol is millimole; M is molar; TFA is trifluoroacetic acid.

The compounds of this invention can be prepared employing the following general procedures. Benzisoxazole intermediates may be prepared from commercially available or readily accessible resorcinols as shown in scheme I or alternate synthetic pathways as reported in the literature. See for example: Shutske, G. M. et al. J. Med. Chem., 25 (1), 36, (1982); Poissonnet, G. Synth. Commun., 27 (22), 3839-3846, (1997); Crabbe, P. Villarino, A. Muchowski, J. M. J. Chem. Soc., Perkin Trans 1, 1973, 2220.

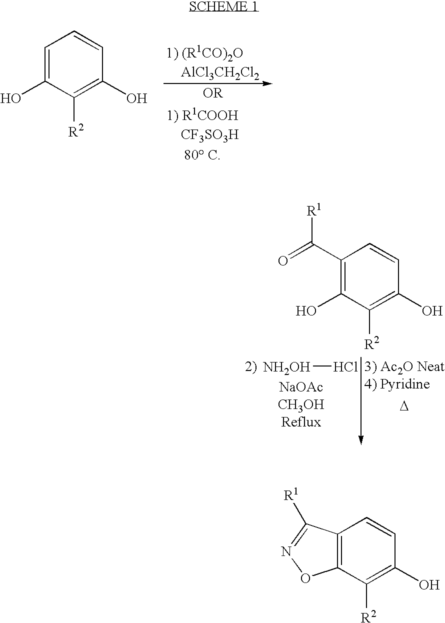

For maximum flexibility these phenolic benzisoxazoles may be converted to intermediate alkylating agents for condensation with a variety of amine substrates, as shown in Scheme 2 below. Alkylation with a more elaborate alkylating agent can lead directly to the desired compounds.

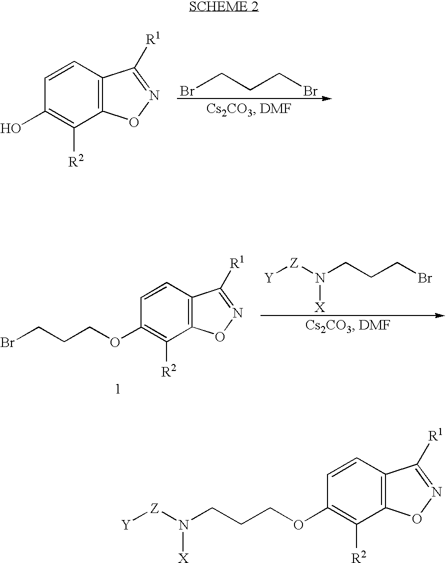

Conversion of the intermediate 1 to the desired amide and imide products can be accomplished by several routes. Examples are given in Schemes 3-6 below for the displacement of the bromide of 1 by an amine followed by acylation of the product primary or secondary amine 2 with an anhydride, cyclic anhydride or acid chloride to give compounds of the general structure in 3a, 3b and 3c. The aryl, alkyl, X and Y groups designated in Schemes 4-6 correspond to the aryl, alkyl, X and Y groups previously defined for compounds of Formula I. In some cases a subsequent hydrolysis or deprotection generates the desired final example.

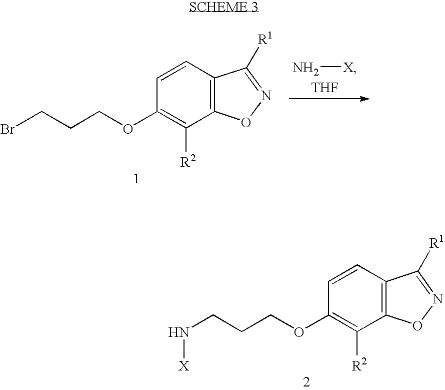

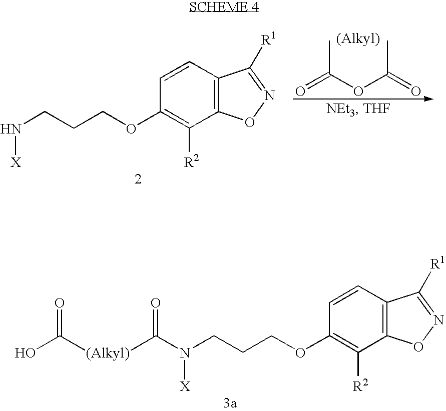

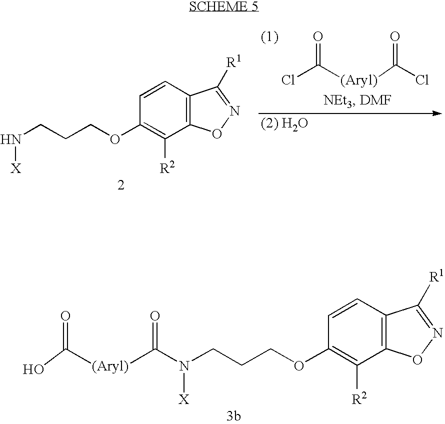

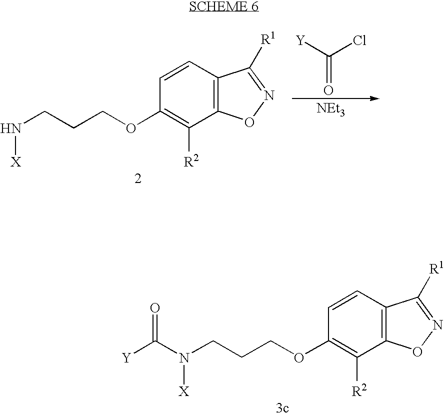

The instant invention provides methods for treating lipid disorders, particularly for treating below-desired plasma HDL cholesterol levels, as well as for treating and/or reducing the risk for diseases and conditions affected by LXR activity, comprising administering a therapeutically effective amount of a compound of Formula I to a person in need of such treatment. Any patient having a depressed plasma HDL cholesterol level, or desiring to increase their HDL cholesterol level may use this treatment. Particularly suitable patients in need of such treatment are those whose plasma HDL cholesterol level is depressed, i.e., below the clinically desirable level. Currently, the clinically desirable HDL cholesterol level is considered to be about 40 mg/dl or higher in men and about 50 mg/dl or higher in women.

The method of this invention also serves to prevent lipid accumulation in, or remove lipids from, tissue deposits such as atherosclerotic plaques or xanthomas in a patient with atherosclerotic disease manifest by clinical signs such as angina, claudication, bruits, one that has suffered a myocardial infarction or transient ischemic attack, or one diagnosed by angiography, sonography or MRI.

Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal, including a human, who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those with dyslipidemic conditions including depressed or below-desirable plasma levels of HDL cholesterol, as well as those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein cholesterol, and a family history of atherosclerotic cardiovascular disease. Published guidelines for determining those who are at risk of developing atherosclerotic disease can be found in: Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp. 2486-2497. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by reverse cholesterol transport.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage amount of a compound of Formual I that a patient receives can be selected so as to achieve the amount of lipid level modification desired, particularly to achieve a desired level of HDL cholesterol. The dosage a patient receives may also be titrated over time in order to reach a target lipid profile. The dosage regimen utilizing a compound of Formula I is selected in accordance with a variety of factors including type, species, age, weight, sex and medical condition of the patient; the severity of the condition to be treated; the potency of the compound chosen to be administered; drug combinations; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

An effective amount of compound for use in the method of this invention is about 0.01 mg/kg to about 30 mg/kg of body weight per day, or about 0.7 mg to about 2100 mg per patient in single or divided doses per day. More particularly, an amount of about 7 mg to about 1050 mg per patient in single or divided doses per day can be administered. However, dosage amounts will vary depending on factors as noted above, including the potency of the particular compound. Although the active drug of the present invention may be administered in divided doses, for example from one to four times daily, a single daily dose of the active drug is preferred.

The active drug employed in the instant therapy can be administered in such oral forms as tablets, capsules, pills, powders, granules, elixirs, tinctures, suspensions, syrups, and emulsions. Oral formulations are preferred.

Administration of the active drug can be via any pharmaceutically acceptable route and in any pharmaceutically acceptable dosage form. This includes the use of oral conventional rapid-release, time controlled-release and delayed-release (such as enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl4-methylphenol (BHT), propyl gallate, sodium ascorbate, citric acid, calcium metabisulphite, hydroquinone, and 7-hydroxycoumarin, can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drug can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolyrners of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

In a broad embodiment, any suitable additional active agent or agents may be used in combination with the compound of Formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), cerivastatin, particularly the sodium salt thereof (see U.S. Pat. No. 5,177,080), pitavastatin also referred to as NK-104 (see PCT international publication number WO 97/23200) and rosuvastatin, also known as ZD-4522, (CRESTOR®; see U.S. Pat. No. 5,260,440, and Drugs of the Future, 1999, 24(5), pp. 511-513); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABCAI gene expression; FXR ligands including both inhibitors and agonists; bisphosphonate compounds such as alendronate sodium; and cyclooxygenase-2 inhibitors such as rofecoxib and celecoxib. Additionally, the compounds of Formula I of this invention, may be used in combination with anti-retroviral therapy in AIDS infected patients to treat lipid abnormalities associated with such treatment, for example but not limited to their use in combination with HV protease inhibitors such as indinavir, nelfinavir, ritonavir and saquinavir.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A—cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors are described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 00/63703, WO /0060107, WO 00/38725, WO 00/34240, WO 00/20623, WO 97/45406, WO 97/16424, WO 97/16455, and WO 95/08532, the entire contents of all of which are hereby incorporated by reference.

An exemplary cholesterol absorption inhibitor is ezetimibe, also known as SCH-58235, which is 1-(4-fluorophenyl)-3(R)-[3(S)-(4-fluorophenyl)-3-hydroxypropyl)]4(S)-(4-hydroxyphenyl)-2-azetidinone, described in U.S. Pat. Nos. 5,767,115 and 5,846,966 and shown below as

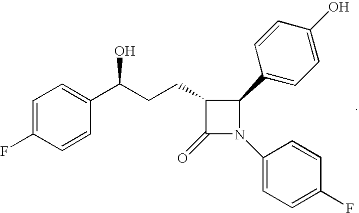

Additional exemplary hydroxy-substituted azetidinone cholesterol absorption inhibitors are specifically described in U.S. Pat. No. 5,767,115, column 39, lines 54-61 and column 40, lines 1-51 (hereby incorporated by reference), represented by the formula

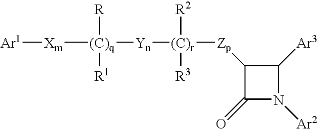

as defined in column 2, lines 20-63 (hereby incorporated by reference). These and other cholesterol absorption inhibitors can be identified according to the assay of hypolipidemic compounds using the hyperlipidemic hamster described in U.S. Pat. No. 5,767,115, column 19, lines 47-65 (hereby incorporated by reference), in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.01 mg/kg to about 30 mg/kg of body weight per day, preferably about 0.1 mg/kg to about 15 mg/kg. For an average body weight of 70 kg, the dosage level is therefore from about 0.7 mg to about 2100 mg of drug per day, e.g. 10, 20, 40, 100 or 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention.

A therapeutically or prophylactically effective amount, as appropriate, of a compound of Formula I can be used for the preparation of a medicament useful for treating lipid disorders, particularly for treating depressed HDL cholesterol levels as well as for treating and/or reducing the risk for diseases and conditions affected by agonism of LXR, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 0.7 mg to about 2100 mg of a compound of Formula I, or more particularly about 7 mg to about 1050 mg. The medicament comprised of a compound of Formula I may also be prepared with one or more additional active agents, such as those described supra.

As used herein, the term LXR includes all subtypes of this receptor. The compounds of Formula I are LXR ligands and individually may vary in their selectivity for one or the other of LXRα and LXRβ, or they may have mixed binding affinity for both LXRα and LXRβ. More particularly, the tested compounds included within the scope of this invention have an IC₅₀ less than or equal to 2 μM for at least one of either the LXRα or LXRβ receptors employing the LXR radioligand competition scintillation proximity assays described below in the Example section. Preferred tested compounds of Formula I bind to the human LXRα receptor have an IC₅₀ less than or equal to 300 nM for the LXRα receptor.

Compound A is used in the following assays and has the following structural formula:

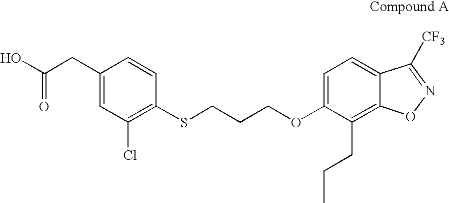

Compound A and related compounds are disclosed along with methods for making them in WO97/28137 herein incorporated by reference in its entirety (U.S. Ser. No. 08/791211, filed Jan. 31, 1997).

The compounds in the following examples were characterized using ¹H NMR at 400 or 500 MHz field strength, and/or by ESI mass spectroscopy (MS).

### EXAMPLE 1

Radioligand Competition Binding Scintillation Proximity Assays:

Preparation of Recombinant Human LXRα and LXRβ:

Human LXRα and LXRβ were expressed as GST-fusion proteins in E. coli. The ligand binding domain cDNAs for human LXRα (amino acids 164447) and human LXRβ (amino acids 149455) were subcloned into the pGEX-KT expression vector (Pharmacia). E. coli containing the respective plasmids were propagated, induced, and harvested by centrifugation. The resuspended pellet was broken in a French press and debris was removed by centrifugation. Recombinant human LXR receptors were purified by affinity chromatography on glutathione sepharose and receptor was eluted with glutathione. Glycerol was added to a final concentration of 50% to stabilize the receptor and aliquots were stored at −80° C.

Binding to LXRα:

For each assay, an aliquot of human GST-LXRα receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.), 0.1% non-fat dry milk and 25 nM [³H₂]Compound A (13.4 Ci/mmole), ±test compound. After incubation for ˜16 h at 15° C. with shaking, the assay plates were counted in a Packard Topcount. In this assay the K_{d} for Compound A for LXRα is ≈15 nM.

Binding to LXRβ:

For each assay, an aliquot of human GST-LXRβ ligand binding domain receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.) 0.1% non-fat dry milk and 25 nM [³H₂]Compound A (13.4 Ci/mmole), ±test compound. After incubation for ˜16 h at 15° C. with shaking, the assay plates were counted in a Packard Topcount. In this assay the K_{d} for Compound A for LXRβ is [symbol] 10 nM.

Results:

Representative tested compounds of Formula I are ligands for human LXRα and/or human LXRβ, each having an IC₅₀ less than or equal to 2,000 nM for the LXRα receptor, and IC₅₀ values ranging from 20 nM to >50,000 nM for the LXRβ receptor.

### EXAMPLE 2

Transactivation Assay

Plasmids

Expression constructs were prepared by inserting the ligand binding domain (LBD) of human LXRα0 and LXRβ cDNAs adjacent to the yeast GALA transcription factor DNA binding domain (DBD) in the mammalian expression vector pcDNA3 to create pcDNA3-LXRα/GAL4 and pcDNA3-LXRβ/GAL4, respectively. The GAL4-responsive reporter construct, pUAS(5X)-tk-luc, contained 5 copies of the GAL4 response element placed adjacent to the thymidine kinase minimal promoter and the luciferase reporter gene. The transfection control vector, pEGFP-N1, contained the Green Fluorescence Protein (GFP) gene under the regulation of the cytomegalovirus promoter.

Assay

HEK-293 cells were seeded at 40,000 cells/well in 96 well plates in Dulbecco's modified Eagle medium (high glucose) containing 10% charcoal stripped fetal calf serum, 100 units/ml Penicillin G and 100 μg/ml Streptomycin sulfate at 37° C. in a humidified atmosphere of 5% CO₂. After 24 h, transfections were performed with Lipofectamine (Gibco-BRL, Gaithersburg, Md.) according to the instructions of the manufacturer. In general, transfection mixes contained 0.002 μg of LXRα/GAL4 or LXRβ/GAL4 chimeric expression vectors, 0.02 μg of reporter vector pUAS(5X)-tk-luc and 0.034 μg of pEGFP-N1 vector as an internal control of transfection efficiency. Compounds were characterized by incubation with transfected cells for 48 h across a range of concentrations. Cell lysates were prepared from washed cells using Cell Lysis Buffer (Promega) according to the manufacturer's directions. Luciferase activity in cell extracts was determined using Luciferase Assay Buffer (Promega) in a ML3000 luminometer (Dynatech Laboratories). GFP expression was determined using the Tecan Spectrofluor Plus at excitation wavelength of 485 nm and emission at 535 nm. Luciferase activity was normalized to GFP expression to account for any variation in efficiency of transfection.

Results with representative tested compounds of Formula I for LXRα transactivation are EC₅₀ of 3 to 3,000 nM, and results for LXRβ transactivation are EC₅₀ of 3 to >10,000 nM.

### EXAMPLE 3

Induction of ABCA1 mRNA levels

Human Primary Macrophages were used to test the ability of LXR ligands to induce the expression of ABCA1 mRNA. The collection and purification of monocytes and their subsequent differentiation into macrophages by culturing in Teflon jars for 7-9 days was performed according to Wright and Silverstein J. Exp. Med. V156, October 1982 pp. 1149-1164. Cells were harvested from the Teflon jars and seeded into appropriate vessels in RPMI1640 plus 12% human serum and antibiotics. Cells were allowed to recover overnight before treatment. Treatment was for 18 hours with the described compounds, followed by a re-application of fresh compounds for an additional 6 hours in DMEM minus Phenol Red with 10% charcoal stripped FCS. The cells were harvested and total RNA prepared using the phenol/guanidine isothiocyanate method as supplied and described by Molecular Research Center, Inc. (TRI REAGENT® Cat. No. TR 118). ABCA1 mRNA levels in the total RNA were measured using the TaqMan® mRNA quantitation system, following protocols published by the manufacturer (Perkin-Elmer). The oligonucleotide PCR primers used to detect ABCA1 were:

GAGGCTCCCGGAGTTGTTG
and
GTATAAAAGAAGCCTCCGAGCATC

The oligonucleotide probe used was:

6FAM-AAACTTTAACAAATCCATTGTGGCTCGCCTGT-TAMRA

ABCA1 mRNA levels in each sample were normalized to the mRNA levels for the 23 kDa highly basic protein. The oligonucleotide PCR primers used to detect the 23 kDa highly basic protein were:

GCTGGAAGTACCAGGCAGTGA
and
ACCGGTAGTGGATCTTGGCTTT

The oligonucleotide probe used was:

VIC-TCTTTCCTCTTCTCCTCCAGGGTGGCT-TAMRA

| | Fold Induction of | |
|---|---|---|
| | ABCA1 mRNA | |
| Compound | (Mean ± SEM) | p Value vs DMSO Control |
| 10 μM 22-(R)- | 8 ± 1.37 | 0.007 |
| hydroxycholesterol | | |
| Compound (3d) 0.1 μM | 0.9 ± 0.2 | 0.93 |
| Compound (3d) 1.0 μM | 3.3 ± 0.3 | 0.002 |
| Compound (3d) 10.0 μM | 13.6 ± 1.1 | 0.0004 |

### EXAMPLE 4

### Step 1 Preparation of 2,4-dihydroxy-3-propyl-1′,1′,1′-trifluoroacetophenone

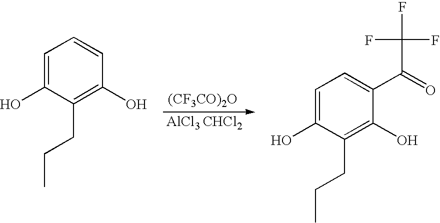

A solution of 2-propylresorcinol (5.0 grams) and trifluoroacetic anhydride (9.6 mL) in 1,2-dichloroethane (30.0 mL) was treated with aluminum chloride (4.38 grams). This mixture was stirred overnight. The reaction mixture was partitioned between methylene chloride and water. The organic phase was dried over sodium sulfate and filtered. The solvent was evaporated and the resulting solid was recrystallized from methylene chloride and cyclohexane (1:1) to give the titled compound.

Selected Signals: ¹H NMR (CDCl₃) δ 7.59 (d, 1H), 6.24 (d, 1H), 5.92 (s, 1H), 2.63 (t, 2H), 1.74 (s, 1H), 1.58 (m, 2H), 0.98 (t, 3H).

### Step 2 Preparation of 3-Trifluoromethyl-7-propyl-6-hydroxybenzisoxazole

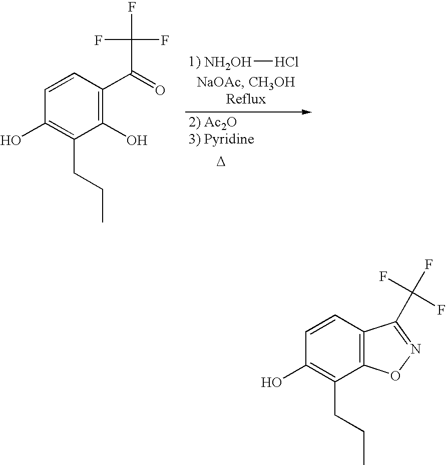

A mixture of 2,4-dihydroxy-3-propyl-1′,1′,1′-trifluoroacetophenone (2.5 grams), sodium acetate (4.18 grams), hydroxylamine hydrochloride (3.59 grarns) and methanol (80 mL) was heated under reflux overnight. The solvent was then evaporated and the resulting solid was partitioned between ethyl acetate and pH 7 buffer. The organic phase was separated and washed with brine. The organic phase was dried over sodium sulfate and the solvent was evaporated to give an oil. The oil was then dissolved in acetic anhydride. The solution was stirred for two hours, then the acetic anhydride was evaporated in vacuo. The residue was partitioned between ethyl acetate and pH 7 buffer and the organic phase was dried over sodium sulfate. The organic phase was evaporated to give an oil. The oil was dissolved in pyridine and refluxed overnight. The solvent was evaporated in vacuo to give an oil which was chromatographed on silica gel using ethyl acetate and hexane (1:4) to give the titled compound.

Selected Signals: ¹H NMR (CDCl₃) δ 7.46 (d, 1H), 6.92 (d, 1H), 5.42 (bs, 1H), 2.89 (t, 2H), 1.74 (m, 2H), 0.98 (t, 3H).

### EXAMPLE 5

### Preparation of 6-Hydroxy-3-neopentyl-7-propyl-1,2-benzisoxazole

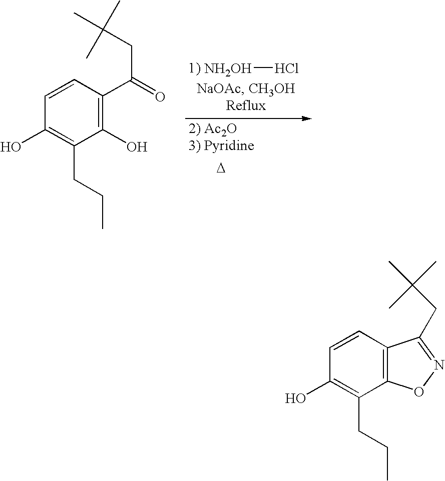

1-(2,4-dihydroxy-3-propylphenyl)-3,3-dimethylbutan-1-one (200 grams, 0.8 mole), prepared as in Example 4 Step 1, was converted to 6-Hydroxy-3-neopentyl-7-propyl-1,2-benzisoxazole, as described in Example 4 Step 2, using hydroxylamine hydrochloride ( 278 grams, 4 mole) and sodium acetate (320 grams) and refluxing in methanol (2.5 L). A second addition of hydroxylamine hydrochloride (106 grams, 1.5 mole) and sodium acetate (250 grams) was made after 18 hours at reflux followed by further heating under reflux for a total of 36 hours. After isolation of the oxime, as described in Example 4 Step 2, the crude material was purified by crystallization from hexanes. Conversion to the oxime acetate was accomplished by dissolving in acetic anhydride, as described in Example 4 Step 2. Full conversion required 18 hours for this case. Ring closure in pyridine, as in Example 4 Step 2, yielded a dark oil. The crude product was eluted from silica gel with methylene chloride. The resulting oil was crystallized from hexanes:ether to yield the titled compound.

Selected Signals: ¹H NMR (CDCl₃) δ 7.33 (d, 1H, J=8.5 Hz), 6.81 (d, 1H, J=8.5 Hz), 5.07 (brd, 1H), 2.89 (collapsed dd, 2H), 1.77 (sect, 2H, J=7.5 Hz), 1.08 (s, 9H), 1.04 (t, 3H, J=7.3 Hz).

### EXAMPLE 6

### Step 1 Preparation of 2,4-Dihydroxy-3-allylbenzophenone

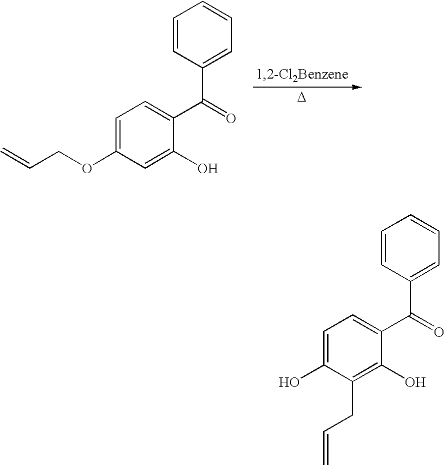

Commercially available 4-allyloxy-2-hydroxybenzophenone (15 grams) was rearranged by heating under reflux in ortho-dichlorobenzene (60 mL) for 26 hours. The product was isolated by dilution of the reaction mixture with 5 volumes hexanes to give the titled compound.

Selected Signals: ¹H NMR (CDCl₃); δ 7.62-7.59 (m, 2H), 7.56-7.52 (m, 2H), 7.49-7.44 (m, 2H), 7.40 (d, 1H, J=8.9 Hz), 6.34 (d, 1H, J=8.8 Hz), 6.02 (ddt, 1H, J=17.21, 10.1, 6.2 Hz), 5.72 (s, 1H, phenol OH), 5.14-5.24 (m, 2H), 3.53 (d with fine splitting, 2H, J=6.2 Hz).

### Step 2 Preparation of 2,4-Dihydroxy-3-propylbenzophenone

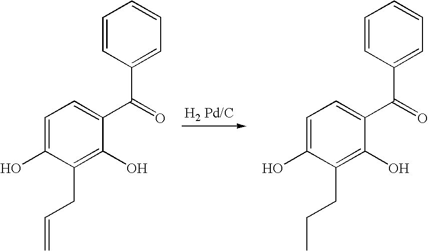

A solution of 2,4-dihydroxy-3-(2-propenyl)benzophenone (3 grams) was reduced under ˜1 atmosphere of H₂ in ethyl acetate (100 mL) over 10% Pd/C catalyst (0.3 grams) for 3 hours. The product was purified by crystallization from methanol/water to give the titled compound.

Selected Signals: ¹H NMR (CDCl₃); δ 7.61-7.59 (m, 2H), 7.55-7.51 (m, 1H), 7.48-7.44 (m, 2H), 7.33 (d, 1H, J=8.8 Hz), 6.29 (d, 1H, J=8.8 Hz), 5.51 (s, 1H, phenol OH), 2.66 (dd, 2H, J=7.6, 9.3 Hz), 1.61 (sext, 2H, J=7.7 Hz), 0.99 (t, 3H, J=7.3 Hz).

### Step 3 Preparation of 6-hydroxy-7-propyl-3-phenylbenzisoxazole

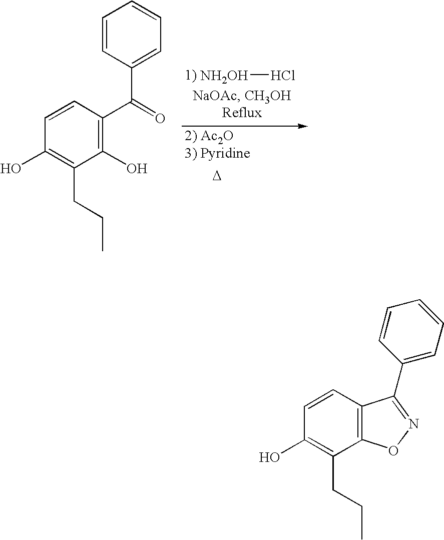

The 2,4-dihydroxy-3-propylbenzophenone (2.5 grams, 9.8 mmol) was converted to the oxime with hydroxylamine hydrochloride (2.7 grams, 39 mmol) and sodium acetate (3.21 grams, 39 mmol), as described in Example 4 Step 2. The oxime was purified by elution from a silica gel column with 97:3 toluene:ethyl acetate. The product oxime (1.82 grams) was further treated, as in Example 4 Step 2, with acetic anhydride (15 mL) and subsequently heated under reflux in pyridine (15 mL). The cooled reaction mixture was poured into 2 N hydrochloric acid and ethyl acetate. The aqueous phase was extracted with ethyl acetate and washed with saturated aqueous sodium bicarbonate, followed by saturated aqueous sodium chloride. The ethyl acetate extracts were dried over sodium sulfate and reduced in vacuo. The residue was taken up in refluxing toluene (50 mL) and cooled to RT to give the titled compound.

Selected Signals: ¹H NMR (CDCl₃); δ 7.92-7.89 (m, 2H), 7.57 (d, 1H, J=8.5 Hz), 7.55-7.49 (m, 3H), 6.86 (d, 1H, J=8.6 Hz), 5.14 (s, 1H, phenol OH), 2.90 (dd, 2H, J=8.9, 7.6 Hz), 1.76 (sext, 2H, J=7.5 Hz), 1.01 (t, 3H, J=7.3 Hz). MS CI NH₃ M+1 254.1

### EXAMPLE 7

### Preparation of 7-propyl-3-(trifluoromethyl)-6-(3-bromopropyloxy)- 1,2-benzisoxazole

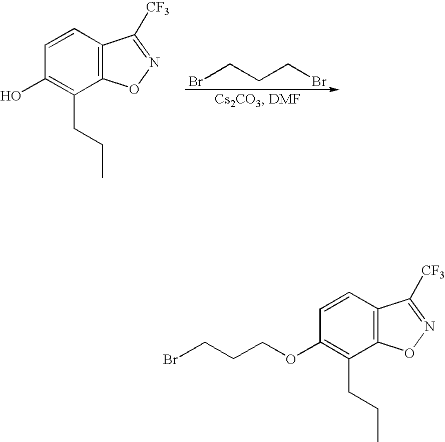

To a DMF solution (50 mL) of 6-hydroxy-7-propyl-3-(trifluoromethyl)-1,2-benzisoxazole as prepared in Example 4 step 2 (5 grams, 20.4 mmol) was added 1,3-dibromopropane (10 mL, 98.5 mmol), followed by cesium carbonate (10 grams, 30.7 mmol). The mixture was stirred at room temperature overnight. After aqueous ether work-up and silica gel chromatography (hexanes: 2.5% ethyl acetate), the titled compound was obtained.

Selected Signals: ¹H NMR (CDCl₃); δ 7.59 (d, 2H, J=8.8 Hz), 7.10 (d, 2H, J=8.8 Hz), 4.27 (t, 2H, J=5.8 Hz), 3.66 (t, 2H, J=6.4 Hz), 2.93 (t, 2H, J=7.5 Hz), 2.41 (pent, 2 H, J=6.0 Hz), 1.72 (sext, 2H, J=7.5 Hz), 0.99 (t, 3H, J=7.5 Hz).

### EXAMPLE 8

### Preparation of [N-methyl-N-(3-{[7-propyl-3-(trifluoromethyl)-1,2-benzisoxazol-6-yl]oxy}propyl)]isophthalic acid monoamide

Step 1. Preparation of

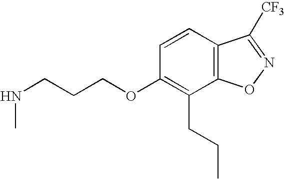

To 1 gram of 3-trifluoromethyl-7-propyl-6-(3-bromopropyloxy)-1,2-benzisoxazole, as prepared in Example 7, was added MeNH₂ (14 mL of a 2 M solution in THF, 14 mmol). The reaction was stirred at room temperature overnight. After concentration in vacuo, the amine was obtained and used directly in the next step.

Step 2. Preparation of

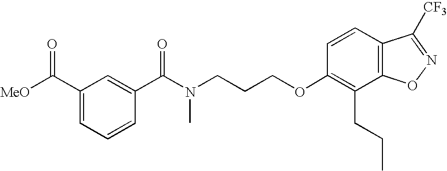

To a solution of the amine from Step 1 in 30 mL of CH₂Cl₂ and 10 mL of DMF was added monomethyl isophthalate (740 mg, 4.1 mmol), HOBt (550 mg, 4.1 mmol), 1-[3-(dimethylamino)propyl]-3-ethylcarbodiimide hydrochloride (790 mg, 4.1 mmol) and i-Pr2NEt (0.72 mL, 4.1 mmol). The mixture was stirred at room temperature overnight. After aqueous ether work-up and chromatography, the methyl ester was obtained.

Step 3. Preparation of [N-methyl-N-(3-{[7-propyl-3-(trifluoromethyl)-1,2-benzisoxazol-6-yl]oxy)propyl)]isophthalic acid monoamide

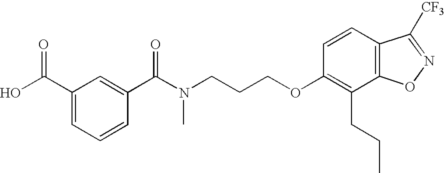

To a solution of the methyl ester from Step 2 (1.1 grams, 2.3 mmol) in MeOH (10 mL), H₂O (10 mL) and THF (5 mL) was added NaOH (11.5 mL of a 1N solution). The mixture was stirred at room temperature overnight, acidified and purified by HPLC to give the titled compound.

Selected Signals: ¹H NMR (CDCl₃): δ 0.8-1.0 ppm (3H, br), 1.5-1.8 (2H, br), 2-2.4 (2H, br), 2.6-3.0 (2H, br), 3.1-3.2 (3H, br), 3.6-3.8 (2H, br), 4.0-4.3 (2H, br), 6.9-8.2 (6H, m).

MS: m/z=465 (M+H).

### EXAMPLE 9

### Preparation of N-methyl-N-(3-{[7-propyl-3-(trifluoromethyl)-1,2-benzisoxazol-6-yl]oxy}propyl)succinic acid monoamide

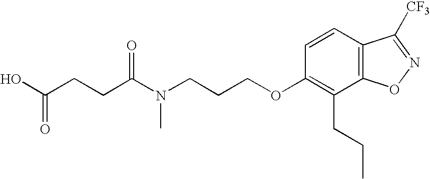

To a solution of the amine from Example 8, Step 1 (20 mg) in THF (2 mL) and DMF (2 mL) was added NEt₃ (18 μL, 0.13 mmol) and succinic anhydride (9.5 mg, 0.095 mmol). The reaction mixture was stirred at 45° C. overnight. After acidification with 50 μL of TFA and HPLC purification, the titled compound was obtained.

Selected Signals: ¹H NMR (CDCl₃): δ 1.00 ppm (3H, t, J=7.5 Hz), 1.75 (2H, m), 2.18 (2H, m), 2.72 (4H, m), 2.94 (2H, m), 3.02-3.12 (3H, 2s), 3.62 (2H, m), 4.15 (2H, m), 7.05 (1H, d, J=8.8), 7.58 (1H, d, J=8.8).

MS: m/z=417 (M+H).

### EXAMPLE 10

### Preparation of 4-carboxy4,4-dimethyl-[N-methyl-N-(3-{[7-propyl-3-(trifluoromethyl)-1,2-benzisoxazol-6-yl]oxy}propyl)]butyramide

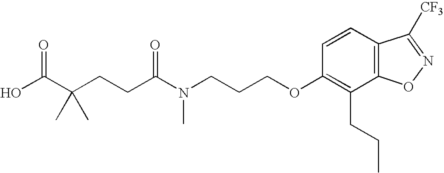

To a solution of the amine from Example 8, Step 1 (20 mg) in THF (2 mL) and DMF (2 mL) was added NEt₃ (18 μL, 0.13 mmol) and 2,2-dimethylglutaric anhydride (13.5 mg, 0.095 mmol). The reaction mixture was stirred at 45° C. overnight. After acidification with 50 μL of TFA and BPLC purification, the titled compound was obtained.

Selected Signals: ¹H NMR (CDCl₃): δ 0.99 ppm (3H, t, J=7.5 Hz), 1.20-1.26 (6H, 2s), 1.70 (2H, m), 1.92 (2H, m), 2.14 (2H, m), 2.38 (2H, m), 2.94 (2H, m), 2.98-3.06 (3H, 2s), 3.60 (2H, m), 4.15 (2H, m), 7.05 (1H, d, J =8.8), 7.58 (1H, d, J=8.8).

MS: m/z=459 (M+H).

### EXAMPLE 11

### Preparation of 4-carboxy-3,3-dimethyl-[N-methyl-N-(3-{[7-propyl-3-(trifluoromethyl)-1,2-benzisoxazol-6-yl]oxy}propyl)]butyramide

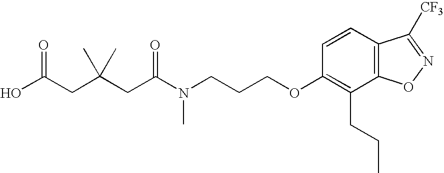

To a solution of the amine from Example 8, Step 1 (1 gram) in THF (20 mL) and DMF (10 mL) was added NEt₃ (0.88 μL, 6.3 mmol) and 3,3-dimethylglutaric anhydride (675 mg, 4.8 mmol). The reaction mixture was stirred at 45° C. overnight. After acidification with 1 mL of TFA and HPLC purification, the titled compound was obtained.

Selected Signals: ¹HNMR (CDCl₃): δ 1.00 ppm (3H, t, J=7.5 Hz), 1.14-1.18 (6H, 2s), 1.73 (2H, m), 2.20 (2H, m), 2.42-2.46 (2H, 2s), 2.32-2.37 (2H, 2s), 2.95 (2H, m), 3.10-3.21 (3H, 2s), 3.72 (2H, m), 4.18 (2H, m), 7.05 (1H, d, J=8.8), 7.60 (1H, d, J=8.8).

MS: m/z=459 (M+H).

### EXAMPLE 12

### Step 1 Preparation of 7-propyl-3-(trifluoromethyl)-6-(3-azidopropyloxy)-1,2-benzisoxazole

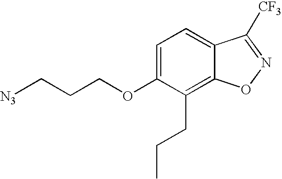

A mixture of 7-propyl-3-(trifluoromethyl)-6-(3-bromopropyloxy)-1,2-benzisoxazole as prepared in Example 7 (8.15 grams, 22.3mmol) and sodium azide (7.24 grams, 111.3 mol) in DMF (100 mL) was stirred at 50° C. for 3 hours. The reaction mixture was partitioned between ether and water. The organic phase was dried over sodium sulfate and filtered. The solvent was evaporated in vacuo to give an oil which was purified by chromatography on silica gel using ethyl acetate and hexane (1:19) to give the titled compound.

Selected Signals: ¹H NMR (CDCl₃) δ 7.37 (d, 1H), 6.92 (d, 1H), 4.09 (t, 2H), 3.41 (t, 2H), 2.86 (t, 2H), 1.93(m, 2H), 1.67 (m, 2H), 0.98 (t, 3H).

MS: m/z=329 (M+H)

### Step 2 Preparation of 7-propyl-3-(trifluoromethyl)-6-(3-aminopropyloxy)-1,2-benzisoxazole

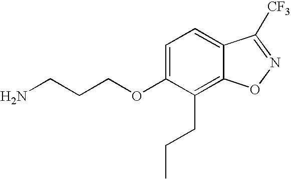

A mixture of 7-propyl-3-(trifluoromethyl)-6-(3-azidopropyloxy)-1,2-benzisoxazole (1.80 grams, 5.26mmol), triphenyl phosphine (2.07 grams, 7.9 mol) and water (0.95 mL, 52.6 mol) in THF from Step 1 (15 mL) was stirred at room temperature for 3 hours. The reaction mixture was partitioned between ethyl acetate and water. The organic phase was dried over sodium sulfate and filtered. The solvent was evaporated and the resulting solid was chromatographed on silica gel using methanol and dichloromethane (1:9) to give the titled compound.

Selected Signals: ¹H NMR (CDCl₃) δ 7.55(d, 1H, J=9.0 Hz), 7.07 (d, 1H, J=9.0), 4.19 (t, 2H, J=6.0), 2.96 (t, 2H, J=6.5), 2.90 (t, 2H, J=7.5), 2.00 (m, 2H), 1.70 (m, 2H), 1.42 (br, 2H), 0.96 (t, 3H, J=5.5).

MS: m/z=303 (M+H)

### EXAMPLE 13

### Preparation of N-(3-{7-propyl-3-(trifluoromethyl)-1,2-benzisoxazole-6-yl]oxy}propyl)acetamide

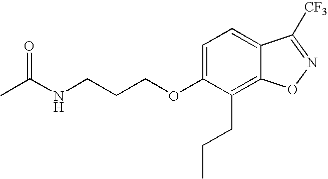

A mixture of 7-propyl-3-(trifluoromethyl)-6-(3-amino propyloxy)-1,2-benzisoxazole as prepared in Example 12, Step 2 (134 mg, 0.44 mmol), triethyl amine (90 mg, 0.89 mmol) and acetyl chloride (35 mg, 0.44 mmol) in methylene chloride (2 mL) was stirred for 3 hours. The reaction mixture was partitioned between ether and water. The organic phase was dried over sodium sulfate and filtered. The solvent was evaporated in vacuo to give an oil which was purified by chromatography on silica gel using methanol and dichloromethane (1:9) to give the titled compound.

Selected Signals: ¹H NMR (CDCl₃) δ 7.56 (d, 1H, J=9.0 Hz), 7.06 (d, 1H, J=9.0), 5.68 (bs, 1H), 4.16 (t, 2H, J =6.0), 3.50 (q, 2H, J=7.0 and 13.0), 2.92 (t, 2H, J=7.0), 2.10(m, 2H), 2.00 (s, 3H), 1.72(m, 2H), 0.98 (t, 3H, J=5.5).

MS: m/z=345 (M+H)

### EXAMPLE 14

### Preparation of N-methyl-N-(3-{[7-propyl-3-(trifluoromethyl)-1.2-benzisoxazol-6-yl]oxy}propyl)acetamide

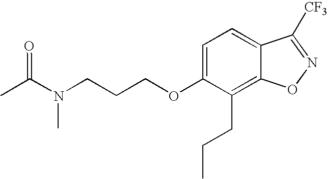

A mixture of N-(3-{7-propyl-3-(trifluoromethyl)-1,2-benzisoxazole-6-yl]oxy}propyl)acetamide as prepared in Example 13 (57.3 mg, 0.17 mmol), sodium hydride (20 mg, 0.50mmol) and methyl iodide (142 mg, 1.0 mmol) in THF (2 mL) was refluxed for 3 hours. The reaction mixture was partitioned between ether and water. The organic phase was dried over sodium sulfate and filtered. The solvent was evaporated in vacuo and was purified by chromatography on silica gel using methanol and dichloromethane (1:16) to give the titled compound.

Selected Signals: ¹H NMR (CDCl₃) δ 7.56 (m, 1H), 7.06 (m, 1H), 4.13 (m, 2H), 3.57 (m, 2H), 3.04& 2.97 ( s, 3H), 2.92 (t, 2H, J =7.5 Hz), 2.10(m, 2H), 2.09(s, 3H), 1.72(m, 2H), 0.98 (m, 3H).

MS: m/z=359 (M+H)

### EXAMPLE 15

### Preparation of N-(3-{7-propyl-3-(trifluoromethyl)-1,2-benzisoxazole-6-yl]oxy}propyl)propionamide

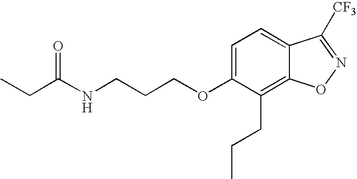

A mixture of 7-propyl-3-(trifluoromethyl)-6-(3-amino propyloxy)-1,2-benzisoxazole as prepared in Example 12, Step 2 (124 mg, 0.41 mmol), triethyl amine (83 mg, 0.82 mmol) and propionyl chloride (38 mg, 0.41 mmol) in methylene chloride (2 mL) was stirred for 3 hours. The reaction mixture was partitioned between ether and water. The organic phase was dried over sodium sulfate and filtered. The solvent was evaporated in vacuo to give an oil which was purified by chromatography on silica gel using methanol and dichloromethane (1:9) to give the titled compound.

Selected Signals: ¹H NMR (CDCl₃) δ 7.56 (d, 1H, J=9.0 Hz), 7.06 (d, 1H, J=9.0), 5.63 (bs, 1H), 4.16 (t, 2H, J=6.0), 3.49 (q, 2H, J =6.5 and 13.0), 2.92 (t, 2H, J=7.5), 2.22 (q, 2H, J=7.5 and 15.0), 2.10(m, 2H), 1.73(m, 2H), 1.17 (t, 3H, J=7.5), 0.97 (t, 3H, J=8.0).

MS: m/z=359 (M+H)

### EXAMPLE 16

### N-methyl-N-(3-[7-propyl-3-(trifluoromethyl)-1,2-benzisoxazol-6-yl]oxy}propyl)propionamide

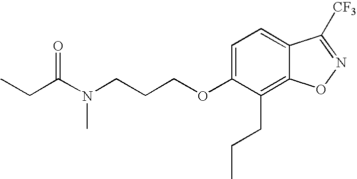

A mixture of N-(3-{7-propyl-3-(trifluoromethyl)-1,2-benzisoxazole-6-yl]oxy}propyl)propionamide as prepared in Example 15 (26.7 mg, 0.07 mmol), sodium hydride (8.9 mg, 0.22 mmol) and methyl iodide (28.0 mg, 0.45 mmol) in THF (2 mL) was refluxed for 3 hours. The reaction mixture was partitioned between ether and water. The organic phase was dried over sodium sulfate and filtered. The solvent was evaporated in vacuo and was purified by chromatography on silica gel using methanol and dichloromethane (1:16) to give the titled compound.

Selected Signals: ¹H NMR (CDCl₃) δ 7.50 (m, 1H), 7.03 (m, 1H), 4.12 (m, 2H), 3.57 (m, 2H), 3.02&2.96 ( s, 3H), 2.90 (m, 2H), 2.36(m, 2H), 2.10 (m, 2H), 1.71 (m, 2H), 1.13 (t,3H), 0.96 (m, 3H).

MS: m/z=373 (M+H)

### EXAMPLE 17

### Preparation of [N-methyl-N-(3-{[7-propyl-3-(trifluoromethyl)-1,2-benzisoxazol-6-yl]oxy}propyl)]thiophene-1,5-dicarboxylic acid monoamide

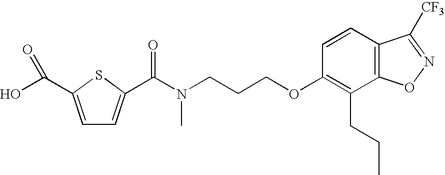

To 5 mL of a 0.1 M solution of the N-methylamine from Example 8, Step 1 (0.05 mmol) in CH₂Cl₂ was added 2,5-thiophenedicarboxylic acid (26.9 mg), HOBT (9.1 mg), EDC HCl (11.9 mg), 2 mL CH₂Cl₂, and then diisopropylethylamine (26.5 μL). The reaction mixture was stirred at room temperature overnight. The solvent was evaporated in vacuo, and the resultant solid was dissolved in 3 mL of methanol and separated by HPLC to give the title compound as a white solid.

Selected Signals: ¹H NMR (CDCl₃); δ 7.77 (b, 1H), 7.58 (d, 1H, J=8.5 Hz), 7.36 (b, 1H), 7.07 (b, 1H), 6.82 (b, 2H), 4.18 (b, 2H), 3.82 (t, 2H, J=7.2 Hz), 3.27 (b, 3H), 2.91 (b, 2H), 2.26 (b, 2H), 1.70 (b, 2H), 0.96 (b, 3H).

MS: m/z=471 (M+H).

### EXAMPLE 18

### Preparation of [N-methyl-N-(3-{[7-propyl-3-(trifluoromethyl)-1,2-benzisoxazol-6-yl]oxy}propyl)]pyridine 3,5-dicarboxylic acid monoamide

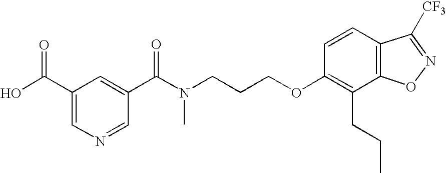

To 5 mL of a 0.1 M solution of the N-methylamine from Example 8, Step 1 (0.05 mmol) in CH₂Cl₂ was added 3,5-pyridinedicarboxylic acid (28.4 mg), HOBT (14.6 mg), EDC HCl (20.2 mg), 2 mL CH₂Cl₂, and then diisopropylethylarnine (26 μL). The reaction mixture was stirred at room temperature overnight. The solvent was evaporated in vacuo, and the resultant solid was dissolved in 3 mL of methanol and separated by HPLC to give the title compound as a white solid.

Selected Signals: ¹H NMR (CDCl₃); δ 10.50 (b, exchangeable H), 9.35 (major rotational isomer, s, 1H) 9.26 (minor rotational isomer, s, 1H), 9.01 (major rotational isomer, s, 1H), 8.94 (minor rotational isomer, s, 1H), 8.62 (major rotational isomer, s, 1H), 8.45 (minor rotational isomer, s, 1H), 7.60 (major rotational isomer, d, 1H, J=9.0 Hz) 7.56 (minor rotational isomer, d, 1H, J=8.5 Hz), 7.12 (major rotational isomer, d, 1H, J=9.0 Hz), 6.98 (minor rotational isomer, d, 1H, J=8.5 Hz), 4.28 (major rotational isomer, t, 2H, J=5.5 Hz), 4.05 (minor rotational isomer, b, 2H), 3.88 (major rotational isomer, t, 2H, J=6.8 Hz), 3.68 (minor rotational isomer, b, 2H), 3.26 (minor rotational isomer, s, 3H), 3.15 (major rotational isomer, s, 3H), 2.96 (major rotational isomer, t, 2H, J=7.2 Hz), 2.54 (minor rotational isomer, t, 2H, J=7.0 Hz), 2.32 (major rotational isomer, b, 2H), 2.18 (minor rotational isomer, b, 2H), 1.75 (major rotational isomer, sextet, 2H, J=7.3 Hz), 1.51 (minor rotational isomer), sextet, 2H, J=7.3 Hz), 1.00 (major rotational isomer, t, 3H, J=7.3 Hz), 0.82 (minor rotational isomer, t, 3H, J=7.3 Hz).

MS: m/z=466 (M+H).

### EXAMPLE 19

### Preparation of [N-methyl-N-(3-{[7-propyl-3-(trifluoromethyl)-1,2-benzisoxazol-6-yl]oxy}propyl)]2,2-dichlorocyclopropane-1,3-dicarboxylic acid monoamide

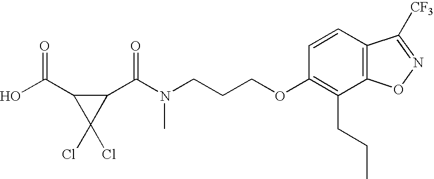

To 5 mL of a 0.1 M solution of the N-methylamine from Example 8, Step 1 (0.05 mmol) in CH₂Cl₂ was added 3,3-dichloro-1,2-cyclopropanedicarboxylic acid (37.3 mg, mixture of cis and trans isomers.), HOBT ( 1.9 mg), EDC HCl (17.2 mg), 2 mL CH₂Cl₂, and then diisopropylethylamine (26 μL). The reaction mixture was stirred at room temperature overnight. The solvent was evaporated in vacuo, and the resultant solid was dissolved in 2 mL of methanol and separated by HPLC to give the title compound as a white solid.

Selected Signals: ¹H NMR (CDCl₃); δ 8.20 (b), 7.58 (minor isomer, d, 1H, J=10.5 Hz), 7.57 (major isomer, d, 1H, J=9.0 Hz), 7.10 (minor isomer, d, 1H, J=9.0 Hz), 7.04 (major isomer, d, 1H, J=8.5 Hz), 4.23 (minor isomer, t, 2H, J=5.5 Hz), 4.15 (major isomer, t, 2H, J=6.0 Hz), 3.96 (minor isomer, m, 1H, J=6.2 Hz), 3.85 (major isomer, m, 1H, J=7.0 Hz), 3.77 (minor isomer, m, 1H), 3.59 (major isomer, m, 1H, J=7.0 Hz), 3.30 (major isomer, s, 3H), 3.18 (minor isomer, s, 3H), 3.12 (d, 2H, J=4.0 Hz), 2.94 (t, 2H, J=7.5 Hz), 2.31 (minor isomer, m, 2H), 2.17 (major isomer, pentet, 2H, J=6.5 Hz), 1.74 (sextet, 2H, J=7.5 Hz), 0.99 (t, 3H, J=7.0 Hz).

MS: m/z=497 (M+H).

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of formula I 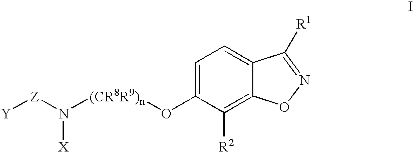
and the pharmaceutically acceptable salts and esters thereof, wherein
R¹ is selected from the group consisting of:
(a) —CF₃,
(b) —CH₂C(CH)₃,
(c) phenyl,
(d) -C₁₋₆ alkyl, and
(e) -C₁₋₂ alkyl-phenyl;
R² is selected from the group consisting of:
(a) -C₁₋₆ alkyl,
(b) —COOR³,
(c) —CR³R⁴—O—R⁵,
(d) —CR³R⁴—S—R⁵, and
(e) —COR³;
R³, R⁴ and R⁵ are independently selected at each occurrence from the group consisting of —H, phenyl, and C₁₋₆ alkyl;
n is an integer selected from 2, 3, 4, 5 and 6;
Z is selected from 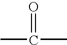
and —S(O)ₘ—, wherein m is an integer selected from zero, 1 and 2;
X is selected from the group consisting of:
(a) —H,
(b) —C(O)CH₃,
(c) -C₁₋₆alkyl, and
(d) —CH₂CF₃;
Y is selected from the group consisting of:
(a) -C₁₋₈ straight chain alkyl, unsubstituted, mono- or poly-substituted with a substituent independently selected at each occurrence from the group consisting of:
(i) —COOR³,
(ii) —CONH₂,
(iii) —CN,
(iv) halo,
(v) thienyl,
(vi) —S-phenyl,
(vii) tetrazolyl,
(viii) NR⁶R⁷,
(ix) phenyl, unsubstituted, mono- or poly-substituted with a substituent independently selected at each occurrence from the group consisting of: —COOR³, halogen and C₁₋₆alkyl,
(x) -C₁₋₃alkyl, unsubstituted, mono- or poly-substituted with a substituent independently selected at each occurrence from the group consisting of: —COOR³, halogen, and -C₁₋₃alkyl,
(b) phenyl, unsubstituted, mono- or poly-substituted with a substituent independently selected at each occurrence from the group consisting of: —COOR³, halogen and -C₁₋₆alkyl,
(c) furanyl, unsubstituted, mono- or poly-substituted with a substituent independently selected at each occurrence from the group consisting of: —COOR³, halogen and -C₁₋₆alkyl,
(d) pyridyl, unsubstituted, mono- or poly-substituted with a substituent independently selected at each occurrence from the group consisting of: —COOR³, halogen and -C₁₋₆alkyl,
(e) thienyl, unsubstituted, mono- or poly-substituted with a substituent independently selected at each occurrence from the group consisting of: —COOR₃, halogen and -C₁₋₆alkyl,
(f) -C₃₋₆cycloalkyl, unsubstituted, mono- or poly-substituted with a substituent independently selected at each occurrence from the group consisting of: —COOR³, halogen and -C₁₋₆alkyl, and
(g) -C₁₋₄alkyl-O-C₁₋₄alkyl, provided that the total number of carbons is from 3 to 5, unsubstituted, unsubstituted, mono- or poly-substituted with a substituent independently selected at each occurrence from the group consisting of: —COOR³ and -C₁₋₆alkyl;
R⁶ is selected from the group consisting of —H and C₁₋₆ alkyl;
R⁷ is selected from the group consisting of:
(a) —COC₁₋₃ alkyl,
(b) —COCF₃, and
(c) —COOR³;
R⁸ and R⁹ are independently selected at each occurrence from the group consisting of —H, C₁₋₄ alkyl, -C₁₋₄ alkenyl, —O-C₁₋₄ alkyl and —O-C₁₋₄ alkenyl; and
provided that when Z is —S(O)ₘ—, then Y is -C₁₋₈ straight chain alkyl.

2. A compound of formula I 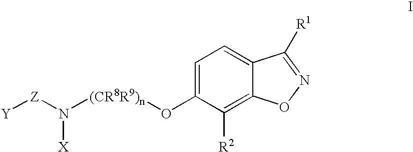
and the pharmaceutically acceptable salts and esters thereof, wherein
R¹ is selected from the group consisting of:
(a) —CF₃,
(b) —CH₂C(CH)₃,
(c) phenyl,
(d) -C₁₋₆ alkyl, and
(e) -C₁₋₂ alkyl-phenyl;
R² is selected from the group consisting of:
(a) -C₁₋₆ alkyl,
(b) —COOR³,
(c) —CR³R⁴—O—R⁵,
(d) —CR³R⁴—S—R⁵, and
(e) —COR³;
R³, R⁴ and R⁵ are independently selected at each occurrence from the group consisting of —H, phenyl, and C₁₋₆ alkyl;
n is an integer selected from 2, 3, 4, 5 and 6;
Z is selected from 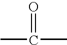
and —S(O)ₘ—, wherein m is an integer selected from zero, 1 and 2;
X is selected from the group consisting of:
(a) —H,
(b) —C(O)CH₃,
(c) -C₁₋₆alkyl, and
(d) —CH₂CF₃;
Y is selected from the group consisting of:
(a) -C₁₋₈ straight chain alkyl, unsubstituted, mono- or poly-substituted with a substituent independently selected at each occurrence from the group consisting of:
(i) —COOR³,
(ii) CONH₂,
(iii) —CN,
(iv) halo,
(v) thienyl,
(vi) —S-phenyl,
(vii) tetrazolyl,
(viii) NR⁶R⁷,
(ix) phenyl, unsubstituted, mono- or poly-substituted with a substituent independently selected at each occurrence from the group consisting of: —COOR³, halogen and C₁₋₆alkyl,
(x) -C₁₋₃alkyl, unsubstituted, mono- or poly-substituted with a substituent independently selected at each occurrence from the group consisting of: —COOR³, halogen, and -C₁₋₃alkyl,
(b) phenyl, unsubstituted, mono- or poly-substituted with a substituent independently selected at each occurrence from the group consisting of: —COOR³, halogen and -C₁₋₆alkyl,
(c) furanyl, unsubstituted, mono- or poly-substituted with a substituent independently selected at each occurrence from the group consisting of: —COOR³, halogen and -C₁₋₆alkyl,
(d) pyridyl, unsubstituted, mono- or poly-substituted with a substituent independently selected at each occurrence from the group consisting of: —COOR³, halogen and -C₁₋₆alkyl,
(e) thienyl, unsubstituted, mono- or poly-substituted with a substituent independently selected at each occurrence from the group consisting of: —COOR³, halogen and -C₁₋₆alkyl,
(f) -C₃₋₆cycloalkyl, unsubstituted, mono- or poly-substituted with a substituent independently selected at each occurrence from the group consisting of: —COOR³, halogen and -C₁₋₆alkyl, and
(g) -C₁₋₄alkyl-O-C₁₋₄alkyl, provided that the total number of carbons is from 3 to 5, unsubstituted, mono- or poly-substituted with a substituent independently selected at each occurrence from the group consisting of: —COOR³ and -C₁₋₆alkyl;
R⁶ is selected from the group consisting of —H and C₁₋₆ alkyl;
R⁷ is selected from the group consisting of:
(a) —COC₁₋₃ alkyl,
(b) —COCF₃, and
(c) —COOR³;
R⁸ and R⁹ are independently selected at each occurrence from the group consisting of —H, C₁₋₄ alkyl, -C₁₋₄ alkenyl, —O-C₁₋₄ alkyl and —O-C₁₋₄ alkenyl;
provided that when Z is —S(O)ₘ—, then Y is -C₁₋₈ straight chain alkyl; and
provided that when Z is —C(O)—, X is H and Y is phenyl substituted with -C₁₋₂ alkyl, then the C₁₋₂ alkyl is not substituted with COOR³ on the terminal carbon.

3. The compound of claim 1 wherein Z is —C(O)—.

4. The compound of claim 3 wherein R¹ is selected from —CF₃ and —CH₂C(CH)₃ and R² is n-propyl.

5. The compound of claim 3 wherein X is selected from —H and C₁₋₆alkyl, and n is selected from 3, 4 and 5.

6. The compound of claim 5 wherein R¹ is selected from —CF₃ and —CH₂C(CH)₃ and R² is n-propyl.

7. The compound of claim 3 wherein Y is selected from
(a) -C₁₋₈ straight chain alkyl, unsubstituted, mono- or poly-substituted with a substituent independently selected at each occurrence from the group consisting of:
(i) —COOR³,
(vii) —CONH₂,
(viii) —CN,
(ix) -halo,
(x) —S-phenyl,
(xi) tetrazolyl, and
(vii) -C₁₋₃alkyl, unsubstituted, mono- or poly-substituted with a substituent independently selected at each occurrence from the group consisting of: —COOR³, halogen, and -C₁₋₃alkyl,
(b) phenyl, unsubstituted, mono- or poly-substituted with a substituent independently selected at each occurrence from the group consisting of: —COOR³, halogen and -C₁₋₆alkyl,
(c) furanyl, unsubstituted, mono- or poly-substituted with a substituent independently selected at each occurrence from the group consisting of: —COOR³, halogen and -C₁₋₆alkyl,
(d) pyridyl, unsubstituted, mono- or poly-substituted with a substituent independently selected at each occurrence from the group consisting of: —COOR³, halogen and -C₁₋₆alkyl,
(e) thienyl, unsubstituted, mono- or poly-substituted with a substituent independently selected at each occurrence from the group consisting of: —COOR³ halogen, and -C₁₋₆alkyl, and
(f) -C₃₋₆cycloalkyl, unsubstituted, mono- or poly-substituted with a substituent independently selected at each occurrence from the group consisting of: —COOR³, halogen and -C₁₋₆alkyl.

8. The compound of claim 7 wherein R¹ is selected from —CF₃ and —CH₂C(CH)₃ and R² is n-propyl.

9. The compound of claim 7 wherein R¹ is selected from —CF₃ and —CH₂C(CH)₃, R² is n-propyl, X is selected from —H, —C(O)CH₃, and C₁₋₃alkyl, R⁸ and R⁹ are independently selected from H and —O-C₁₋₄ alkenyl, and n is selected from 3, 4 and 5.

10. The compound of claim 7 wherein R¹ is —CF₃; R² is n-propyl; X is selected from —H and -C₁₋₂alkyl; n is selected from 3 and 4; R⁸ and R⁹ are H; and Y is selected from
(a) -C₁₋₈ straight chain alkyl, unsubstituted, mono- or poly-substituted with a substituent independently selected at each occurrence from —COOH, —CN, tetrazolyl, and -C₁₋₃alkyl,
(b) phenyl, unsubstituted or mono- or poly-substituted with —COOH,
(c) pyridyl, unsubstituted or mono- or poly-substituted with —COOH,
(d) thienyl, unsubstituted or mono- or poly-substituted with —COOH, and
(e) -C₃₋₆cycloalkyl, unsubstituted, mono- or poly-substituted with a substituent independently selected at each occurrence from —COOH and halogen.

11. The compound of claim 7 wherein R¹ is —CF₃; R² is n-propyl; n is 3; X is methyl; R⁸ and R⁹ are H; and Y is selected from
(a) -C₁₋₈ straight chain alkyl, unsubstituted, mono- or poly-substituted with a substituent independently selected at each occurrence from —COOH and -C₁₋₃alkyl,
(d) phenyl, unsubstituted or mono- or poly-substituted with —COOH, and
(e) -C₃₋₆cycloalkyl, unsubstituted, mono- or poly-substituted with a substituent independently selected at each occurrence from —COOH and halogen.

12. The compound of claim 7 selected from the group consisting of
[N-methyl-N-(3-{[7-propyl-3-(trifluoromethyl)-1,2-benzisoxazol-6-yl]oxy}propyl)]isophthalic acid monoamide;
N-methyl-N-(3-{[7-propyl-3-(trifluoromethyl)-1,2-benzisoxazol-6-yl]oxy}propyl)succinic acid monoamide;
4-carboxy-3,3-dimethyl-[N-methyl-N-(3-{[7-propyl-3-(trifluoromethyl)-1,2-benzisoxazol-6-yl]oxy}propyl)]butyramide;
N-methyl-N-(3-{[7-propyl-3-(trifluoromethyl)-1,2-benzisoxazol-6-yl]oxy}propyl)acetamide;
[N-methyl-N-(3-{[7-propyl-3-(trifluoromethyl)-1,2-benzisoxazol-6-yl]oxy}propyl)]thiophene-1,5-dicarboxylic acid monoamide;
[N-methyl-N-(3-{[7-propyl-3-(trifluoromethyl)-1,2-benzisoxazol-6-yl]oxy}propyl)]pyridine 3,5-dicarboxylic acid monoamide;
[N-methyl-N-(3-{[7-propyl-3-(trifluoromethyl)-1,2-benzisoxazol-6-yl]oxy}propyl)]2,2-dichlorocyclopropane-1,3-dicarboxylic acid monoamide;
and the pharmaceutically acceptable salts and esters thereof.

13. The compound of claim 12 wherein the compound is [N-methyl-N-(3-{[7-propyl-3-(trifluoromethyl)-1,2-benzisoxazol-6-yl]oxy}propyl)]isophthalic acid monoamide and the pharmaceutically acceptable salts and esters thereof.

14. The compound of claim 12 wherein the compound is N-methyl-N-(3-{[7-propyl-3-(trifluoromethyl)-1,2-benzisoxazol-6-yl]oxy}propyl)succinic acid monoamide and the pharmaceutically acceptable salts and esters thereof.

15. The compound of claim 12 wherein the compound is 4-carboxy-3,3-dimethyl-[N-methyl-N-(3-{[7-propyl-3-(trifluoromethyl)-1,2-benzisoxazol-6-yl]oxy}propyl)]butyramide and the pharmaceutically acceptable salts and esters thereof.

16. The compound of claim 1 wherein Z is —S(O)ₘ—.

17. The compound of claim 16 wherein m is 2.

18. A method for treating dyslipidemia comprising administering a therapeutically effective amount of a compound of claim 1 to a patient in need thereof.

19. The method of claim 18 wherein the dyslipidemia comprises depressed plasma HDL cholesterol level.

20. A method for treating atherosclerosis comprising administering a therapeutically effective amount of a compound of claim 1 to a patient in need thereof.

21. A method for reducing the risk of occurrence of atherosclerosis comprising administering a prophylactically effective amount of a compound of claim 1 to a patient at risk for developing atherosclerosis.

22. A method for reducing the risk of occurrence of an atherosclerotic disease event comprising administering a prophylactically effective amount of a compound of claim 1 to a patient at risk for having an atherosclerotic disease event.

23. A method for slowing the progression of atherosclerotic disease comprising the administration of a therapeutically effective amount of a compound of Formula I to a patient who has atherosclerotic disease.

24. A method for removing cholesterol from tissue deposits comprising administering a therapeutically effective amount of a compound of claim 1 to a patient in need thereof.

25. A method for preventing lipid accumulation in tissue deposits comprising administering a prophylactically effective amount of a compound of claim 1 to a patient in need thereof.

26. A pharmaceutical composition comprised of a compound of claim 1 and a pharmaceutically acceptable carrier.

27. A pharmaceutical composition made by combining a compound of claim 1 with a pharmaceutically acceptable carrier.

28. A process for preparing a pharmaceutical composition comprising combining a compound of Formula I with a pharmaceutically acceptable carrier.
